SEQUENCE LISTING (14 sequences)

SEQ ID NO: 1 - protein, 106 aa, Artificial Sequence
  combinatorial chimeric antigen receptor
MTQTPLSLPVSLGDQASISCRSSQTIVHSNGHTYLEWYLQKPGQSPKLLIYKVSNRFSGVPDRFSGSGSGTDFTLKISRVEAEDLGIYYCFQGSHVPYTFGGGTKL

SEQ ID NO: 2 - protein, 138 aa, Artificial Sequence
  combinatorial chimeric antigen receptor
LVQSGPELKKPGETVKISCKASGYTFTNYGMHWVKQAPGKGLKWMGWINTYTGEPTYADDFKGRFAFSLETSASTAYLQINNLKNEDMATYFCARGGYFVHWYFDVWGAGTTVTVSSDPFVPVFLPAKPTTTPAPRPP

SEQ ID NO: 3 - protein, 112 aa, Artificial Sequence
  combinatorial chimeric antigen receptor
DVLMTQTPLSLPVSLGDQASISCRSSQTIVHSNGHTYLEWYLQKPGQSPKLLIYKVSNRFSGVPDRFSGSGSGTDFTLKISRVEAEDLGIYYCFQGSHVPYTFGGGTKLEIK

SEQ ID NO: 4 - protein, 144 aa, Artificial Sequence
  combinatorial chimeric antigen receptor
QIQLVQSGPELKKPGETVKISCKASGYTFTNYGMHWVKQAPGKGLKWMGWINTYTGEPTYADDFKGRFAFSLETSASTAYLQINNLKNEDMATYFCARGGYFVHWYFDVWGAGTTVTVSSDPFVPVFLPAKPTTTPAPRPPTPA

SEQ ID NO: 5 - protein, 11 aa, Artificial Sequence
  combinatorial chimeric antigen receptor
QTIVHSNGHTY

SEQ ID NO: 6 - protein, 4 aa, Artificial Sequence
  combinatorial chimeric antigen receptor
  misc_feature (4)..(4): Xaa can be any naturally occurring amino acid
KVSX

SEQ ID NO: 7 - protein, 11 aa, Artificial Sequence
  combinatorial chimeric antigen receptor
CFQGSHVPYTF

SEQ ID NO: 8 - protein, 8 aa, Artificial Sequence
  combinatorial chimeric antigen receptor
GYTFTNYG

SEQ ID NO: 9 - protein, 8 aa, Artificial Sequence
  combinatorial chimeric antigen receptor
INTYTGEP

SEQ ID NO: 10 - protein, 15 aa, Artificial Sequence
  combinatorial chimeric antigen receptor
CARGGYFVHWYFDVW

SEQ ID NO: 11 - protein, 21 aa, Artificial Sequence
  combinatorial chimeric antigen receptor
IYIWAPLAGTCGVLLLSLVIT

SEQ ID NO: 12 - protein, 113 aa, Artificial Sequence
  combinatorial chimeric antigen receptor
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 13 - protein, 42 aa, Artificial Sequence
  combinatorial chimeric antigen receptor
KRGRKKLLYIFKQPFMRPVQTTQEEDGCSCRFPEEEEGGCEL

SEQ ID NO: 14 - protein, 271 aa, Artificial Sequence
  combinatorial chimeric antigen receptor
METDTLLLWVLLLWVPGSTGDVLMTQTPLSLPVSLGDQASISCRSSQTIVHSNGHTYLEWYLQKPGQSPKLLIYKVSNRFSGVPDRFSGSGSGTDFTLKISRVEAEDLGIYYCFQGSHVPYTFGGGTKLEIKGGGSGGGGSGGGGSGGGGSQIQLVQSGPELKKPGETVKISCKASGYTFTNYGMHWVKQAPGKGLKWMGWINTYTGEPTYADDFKGRFAFSLETSASTAYLQINNLKNEDMATYFCARGGYFVHWYFDVWGAGTTVTVSS

(19) United States
(12) Patent Application Publication
(10) Pub. No.: US 2022/0177573 A1
(43) Pub. Date: Jun. 9, 2022

(54) TWO CHIMERIC ANTIGEN RECEPTORS SPECIFICALLY BINDING CD19 AND IGKAPPA

(71) Applicant: Oslo Universitetssykehus HF, Oslo (NO)

(72) Inventors: Gunnar Kvalheim, Oslo (NO); Erlend Smeland, Oslo (NO); Else Marit Inderberg, Oslo (NO); Sébastien Wälchli, Oslo (NO); Harald Holte, Oslo (NO); June Helen Myklebust, Asker (NO); Steinar Funderud, Oslo (NO); Hakan Köksal, Oslo (NO)

(21) Appl. No.: 17/258,238

(22) PCT Filed: Jul. 8, 2019
(86) PCT No.: PCT/EP2019/068244

§ 371 Date: Jan. 6, 2021

(30) Foreign Application Priority Data
Jul. 9, 2018 (NO) 20180963

(51) Int. Cl. C07K 16/28; C07K 16/42; C07K 14/705; C07K 14/725; A61P 35/00; A61K 35/17

(52) U.S. Cl. CPC C07K 16/2803; A61K 38/00; C07K 14/70578; C07K 14/7051; A61P 35/00; A61K 35/17; C07K 14/70521; C07K 14/70596; C07K 2319/30; C07K 2319/33; C07K 2319/03; C07K 2319/02; C07K 2317/565; C07K 2317/524; C07K 2317/526; C07K 16/42

(57) ABSTRACT

The present disclosure relates to compositions comprising compounds or cells able to specifically bind immunoglobulin kappa (IgKappa) and membrane molecule CD 19 under physiological conditions. In particular, the disclosure relates to a combinatorial chimeric antigen receptor (cCAR) with antigen binding domains specific for the antigen CD19 and the immunoglobulin (Ig) Kappa light chain and their expression in immune effector cells to target cells expressing CD19 and IgKappa, and such immune cells for use in treating B-cell cancers.

### CROSS REFERENCE TO RELATED APPLICATIONS

This application is a National Stage application of PCT/EP2019/068244, filed Jul. 8, 2019, which claims the benefit of priority to Norwegian Patent Application No. 20180963, filed Jul. 9, 2018, both of which are incorporated by reference in their entirety herein.

### FIELD OF THE DISCLOSURE

The present disclosure relates to compositions comprising compounds or cells able to specifically bind immunoglobulin kappa light chain (IgKappa) and membrane molecule CD19 under physiological conditions. In particular, the disclosure relates to a combinatorial chimeric antigen receptor (cCAR) with antigen binding domains specific for the antigen CD19 and the immunoglobulin (Ig) Kappa light chain and their expression in immune effector cells to target cells expressing CD19 and IgKappa, and such immune cells for use in treating B-cell cancers. The disclosure provides nucleic acid molecules encoding such CARs and vectors containing them which may be used to modify immune effector cells to express both CARs.

### SEQUENCE LISTING

The Instant Application contains a Sequence Listing which has been submitted electronically in ASCII format and is hereby incorporated by reference in its entirety. Said ASCII copy, created on Jul. 29, 2021 is named “OSA0058US_ST25 (REVISED)” and is 11,109 bytes in size.

### BACKGROUND

Immunotherapy using antibodies, particularly monoclonal antibodies, has emerged in recent years as a safe and selective method for treating cancer and other diseases. Various extracellular cancer antigens have been identified but antibodies developed against a number of antigens expressed on the surface of B-cells, e.g. CD19, CD20 and CD22, have particularly been successful in the treatment of B-cell malignancies.

Kymriah (tisagenlecleucel) and Yescarta (axicabtagene ciloleucel) are recently approved drugs comprising genetically modified autologous T-cells expressing a chimeric antigen receptor (CAR) specific for the B-cell membrane molecule CD19. Since CD19 is a general B-cell antigen, such CAR-T cells can eliminate all B-lineage cells, including nonmalignant B cells. Accordingly, the cells may recognize and eliminate both CD19-expressing malignant as well as normal cells, and there is a risk that the entire B-cell population in the patient may be eradicated during the treatment. If this occurs, the patient will suffer from impaired humoral immune responses, in particular, B-cell aplasia and hypogammaglobulinemia which might increase the susceptibility of severe infections, sometimes leading to death.

B-cell lymphoma and chronic lymphocytic leukemia cells have a clonally restricted expression of Immunoglobulin (Ig) light chains, meaning that they either acquire Ig-kappa or Ig-lambda. Thus, in order to reduce the on-target toxicity induced by CD19 targeting CAR-T cells to improve the life quality of the patient, alternative immunotherapies are explored. An alternative and more gentle approach would be to target B-cells via their B-cell receptors (BCRs). Since individual B cells express BCR with either Ig kappa (κ) light chains or Ig lambda (λ) light chains due to allelic exclusion, it is possible to eliminate a malignant B-cell population expressing BCRs comprising kappa light chains while saving the normal B-cells expressing BCRs comprising lambda light chains. By using such an approach, it should be possible to avoid impaired humoral immune responses in the patients.

Some prior art documents have suggested the use of chimeric antigen receptors (CARs) binding IgKappa without disclosing the sequences (Ramos et al 2016 and Vera et al 2006). However, US20170049819 (Bluebird) concerns CARs binding kappa light chain and provides a sequence. US' 819 contemplates a type of cellular therapy wherein T-cells are genetically modified to express a CAR targeting malignant B-cells that express a K or 2 light chain polypeptide, and the CAR T-cell is infused to a recipient in need thereof. Unlike antibody therapies, CAR T-cells may be able to replicate in vivo resulting in long-term persistence that can lead to sustained cancer therapy. Only a single CAR sequence is provided in US′819, thus there is still a need for alternative sequences able to form an antigen binding protein specific for IgKappa under physiological conditions. Any alternative may prove useful if undesired immunologic responses are connected to the therapeutic use of the prior art antigen binding proteins.

WO2016172703 (Haemalogix) discloses an alternative that is concerned with Kappa myeloma antigen chimeric antigen receptors and uses thereof. However, the CARs therein bind to a conformational epitope in the switch region of human kappa light chain that is only available when the kappa chain is not associated with a heavy chain. Accordingly, they will not bind to intact kappa-chain containing IgGs or BCRs.

### SUMMARY

It is found that soluble IgGs may reduce the cytotoxicity of the immune effector cells expressing a CAR specific for IgKappa. However, by expressing a CAR specific for IgKappa together with a CAR specific for CD19, this problem may be at least partly avoided. Thus, the present disclosure provides immune effector cells expressing a CAR specific for IgKappa and a CAR specific for CD19 in their cell membrane. Such cells may provide significant cytotoxicity while keeping specificity for IgKappa positive B-cells even in the presence of soluble IgGs. In particular, T cells expressing a CAR specific for IgKappa comprising a CD3ζ-signaling domain and further expressing a CAR specific for CD19 comprising a 4-1BB signaling domain, may provide specific cytotoxicity for IgKappa positive B cells. Accordingly, such cells may thus provide therapeutic effect via toxicity to a clonal population of IgKappa positive B-cells even in presence of serum IgGs.

Accordingly, immune effector cells expressing both these types of CARs may be an improved alternative with respect to specificity compared to therapy based on a single CAR specific for IgKappa only. Furthermore, immune effector cells co-expressing these types of CARs may be an improved alternative based on cytotoxicity compared to conventional therapy with a single CAR specific for CD19 only.

The present disclosure also provides an antigen binding protein specific for human IgKappa under physiological conditions. The antigen binding protein can be used for many purposes, e.g. to construct a CAR which, when expressed in the cell membrane of immune effector cells, provides specific cytotoxicity to B-cells expressing IgKappa. Such CAR may comprise an extracellular domain comprising the antigen binding protein specific for IgKappa under physiological conditions, a transmembrane domain and an intracellular domain able to trigger an immune response upon binding of IgKappa.

In a first embodiment, the present disclosure concerns a cytotoxic immune cell expressing at least two CARs in the cell membrane: a CAR specific for CD19 comprising an extracellular domain, a transmembrane domain and an intracellular costimulatory domain; and a CAR specific for IgKappa comprising an extracellular domain, a transmembrane domain and an intracellular signaling domain In one aspect, the intracellular domain of the CAR specific for CD19 does not comprise a functional intracellular signaling domain (“signal 1” domain). In one aspect, the intracellular domain of the CAR specific for CD19 does not comprise a functional CD3ζ-signaling domain. In another aspect, the intracellular domain of the CAR specific for IgKappa does not comprise a functional costimulatory domain (“signal 2” domain) In another aspect the intracellular domain of the CAR specific for CD19 comprises or consists of a 4-1BB signaling domain (19BB); and the intracellular domain of the CAR specific for IgKappa comprises or consists of a CD3ζ-signaling domain (Kz).

In a second embodiment, the present disclosure concerns a pharmaceutical composition suitable for intravenous, intraperitoneal or subcutaneous administration comprising a therapeutic amount of the cells according to the first embodiment. Said cytotoxic immune cell can be used as a medicament, in particular for treatment of B-cell cancers.

In a third embodiment, the present disclosure concerns nucleic acids encoding the CARs herein, in particular the cCARs.

### BRIEF DESCRIPTION OF THE FIGURES

FIG. 1 shows the sequence of a scFv-fragment specific for IgKappa and the structure of a CAR specific for IgKappa wherein the intracellular domain comprises both “signal 1” and “signal 2” domains (IGK). In the scFv-fragment, the L-chain is in bold font, the VL-chain contains three CDRs boxed, the glycine-serine linker is underlined, the VH-chain contains three CDRs boxed.

FIG. 2 shows flow cytometry plots of T cells not transduced or transduced with the indicated CAR constructs. CAR constructs are detected with specific antibodies (anti-Fab) or binding proteins (Protein L).

FIG. 3a shows the IgKappa and IgLambda expression profile of various cell lines and FIG. 3b shows killing assays of T-cells expressing IGK CAR against previously profiled cell lines.

FIG. 4a illustrates the design of a combinatorial CARs according to the disclosure, with two scFvs, one that is specific for CD19 and one specific for IgKappa.

FIG. 4b demonstrates that presence of serum purified IgGs inhibits the cytotoxicity of T-cells expressing a CAR specific for IgKappa. The black closed circles connected with a line represents mock. The dark closed circles connected with a line represents IGK CAR and the light open circles connected with a line represents Kz. T-cells expressing a CAR specific for CD19 were essentially unaffected by the presence of IgGs. The CD19 CAR is represented by light closed squares connected with a line. T-cells expressing combinatorial CARs specific for CD19 and for IgKappa demonstrated similar cytotoxic activity. 19z-KBB CAR, represented by light open circles connected with a dashed line, was not affected by the presence of increasing IgG concentrations whereas Kz-19BB CAR, represented by dark open circles connected with a dashed line, was weakly affected from the higher concentrations.

FIG. 4c illustrates the structure of six CAR constructs, wherein the antigen binding domain, the transmembrane domain (obtained from a CD8α sequence) and the intracellular domains are indicated.

FIG. 5 shows the cytotoxic potentials of CARs against Ig kappa positive and negative cell lines. IGK CAR is only potent against Ig kappa positive target cells (BL-41) and limited cytotoxic activity was observed against IgKappa negative, IgLambda positive cells (Granta-519). The combinatorial CAR Kz-19BB demonstrates a similar selectivity as the IGK CAR. Still as potent against IgKappa positive target cells and less harmful to IgKappa negative cells than CD19 CAR. Additionally, the same CAR constructs were tested in the presence of serum purified immunoglobulins (IgG). IGK CAR T cells were inhibited in the presence of very low concentration of IgG and killing efficiency reduces significantly whereas CD19 CAR was not affected by the presence of soluble IgG at all, proving the effect is specific to IGK CAR T cells. Furthermore, the combinatorial CARs were less affected by the same IgG concentrations, proving that Kz-19BB combinatorial CAR limits the IgG related inhibition by increasing potency through CD19 scFv dependent secondary signal. This suggests that combinatorial CARs may create a balance between potency and specificity. In particular, the cCAR Kz-19BB demonstrates significant cytotoxicity while keeping specificity for IgKappa positive B-cells and maintaining cytotoxic potential even in the presence of soluble IgGs unlike classic IGK CAR.

FIG. 6 shows the cytotoxic potential of the combinatorial CAR Kz-19BB is adjustable by the adjusting the relative expression level of Kz to 19BB. In the presence of high concentration of IgG, classic IGK CAR activity is inhibited. However, the effect is recovered with increasing relative expression of 19-BB. The performance of the combinatorial CAR Kz-19BB is fully adjustable and can be fine tuned by adjusting the expression level of the individual CAR components to obtain an optimal balance between cytotoxic potential and the specificity to eliminate the malignant B-cell portion and hence save IgLambda positive healthy B cells to reduce the harmful impact of classic CD19 CAR T cells on general humoral immune response.

### DEFINITIONS

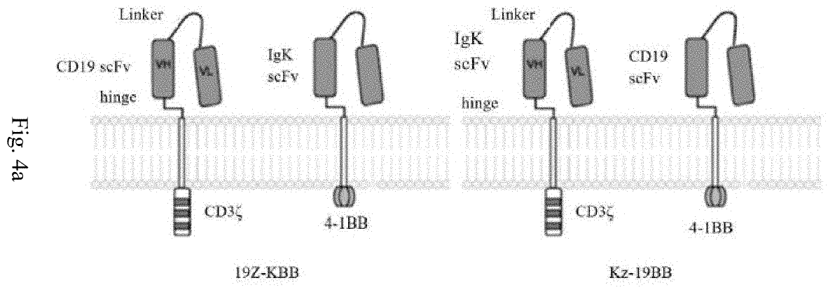

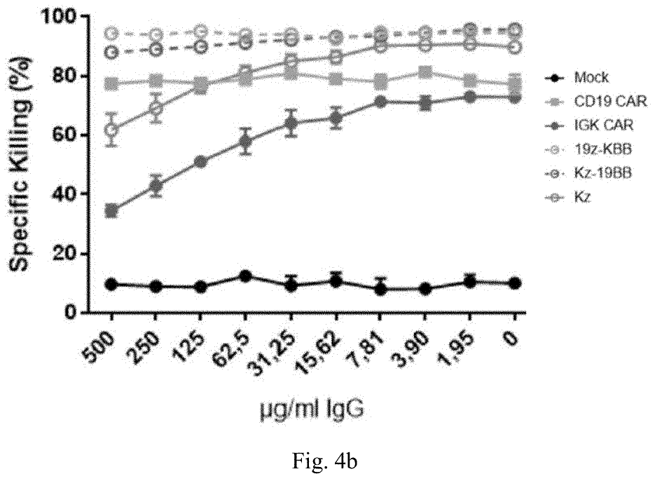

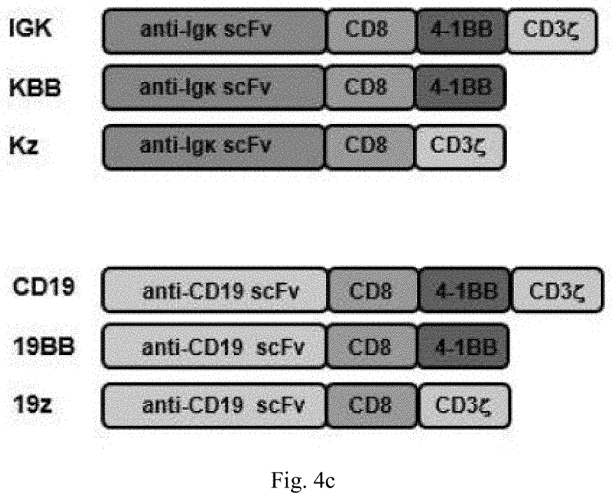

As used herein, a combinatorial chimeric antigen receptor (cCAR) refers to a combination of at least two CARs expressed on the same cells, comprising an antigen binding domain targeting CD19 and an antigen binding domain targeting IgKappa. A cytotoxic immune cell expressing a combinatorial CAR will express at least two different CARs in the cell membrane (illustrated in FIG. 4a). An immune cell expressing a combinatorial CAR according to the disclosure would require simultaneous recognition of both antigens, in order to reach optimal activation status.

As used herein, the antigen-binding (Fab) fragment (or domain) refers to the region of an antibody that binds to antigens. It is composed of one constant and one variable domain of each of the Ig heavy and the light chain. Both chains are encoded by separated genes. The variable domain contains the paratope, comprising a set of complementarities determining regions (CDR), at the amino terminal end of the monomer, which constitute the antigen-binding part.

As used herein, the single-chain variable fragment (scFv) refers to an artificial construct mimicking the antigen binding fragments (Fab) but shorter and encoded by a single coding sequence. An antigen binding fusion protein comprising the variable region of a Ig heavy (VH) and light chain (VL) (and not the constant domains), connected with a short linker peptide of ten to about 25 amino acids, usually (G4S)4 repeat. They are predicted/expected to fold together and reproduce the structure of one arm of the antibody they were designed from.

As used herein, B-cell receptors (BCRs) comprising kappa light chains are referred to IgKappa. The B-lymphoma cell line BL-41 is an example of an IgKappa positive target cell line, meaning a cell line expressing immunoglobulins, e.g. BCRs, which comprises kappa light chains.

As used herein, B-cell receptors (BCRs) comprising lambda light chains are referred to IgLambda. The B-lymphoma cell line Granta-519 is an example of an IgLambda positive target cell line, meaning a cell line expressing immunoglobulins, e.g. BCRs, which comprises lambda light chains.

As used herein, “specific for IgKappa” and “specific for IgLambda” refers to measurable and reproducible interactions with BCRs comprising the kappa light chain and BCRs comprising the lambda light chain, respectively. For example, an antibody comprising an antigen binding domain specific for IgKappa binds its target with greater affinity, avidity, more readily, and/or with greater duration than it binds to other targets. In particular, an antigen binding domain specific for BCRs comprising the kappa light chain will have negligible binding of BCRs comprising the lambda light chain under physiological conditions. Accordingly, T-cells expressing CARs comprising antigen binding domains specific for IgKappa may provide significant killing of IgKappa positive cells, but provide low killing levels when tested on IgLambda positive cells.

As used herein, “specific for CD19” refers to measurable and reproducible interactions with the antigen CD19. For example, an antibody comprising an antigen binding domain specific for CD19 binds its target with greater affinity, avidity, more readily, and/or with greater duration than it binds to other targets.

As used herein, “physiological conditions” means any in vitro or in vivo condition suitable for growth, proliferation, propagation and/or function of human cells, for example neutral aqueous buffer solutions at 37° C.

The term “cytotoxic” is synonymous with “cytolytic” and is used herein to refer to a cell capable of inducing cell death by lysis or apoptosis in a target cell.

The term “immune effector cell” as used herein includes not only mature or fully differentiated immune effector cells but also precursor (or progenitor) cells therefore, including stem cells (more particularly hematopoietic stem cells, HSC), or cells derived from HSC. An immune effector cell may accordingly be a T-cell, NK cell, NKT cell, neutrophil, macrophage, or a cell derived from HSCs contained within the CD19+ population of cells derived from a haemopoietic tissue, e.g. from bone marrow, cord blood, or blood e.g. mobilised peripheral blood, which upon administration to a subject differentiate into mature immune effector cells. As will be described in more detail below, in preferred embodiments, the immune effector cell is a T-cell or an NK cell.

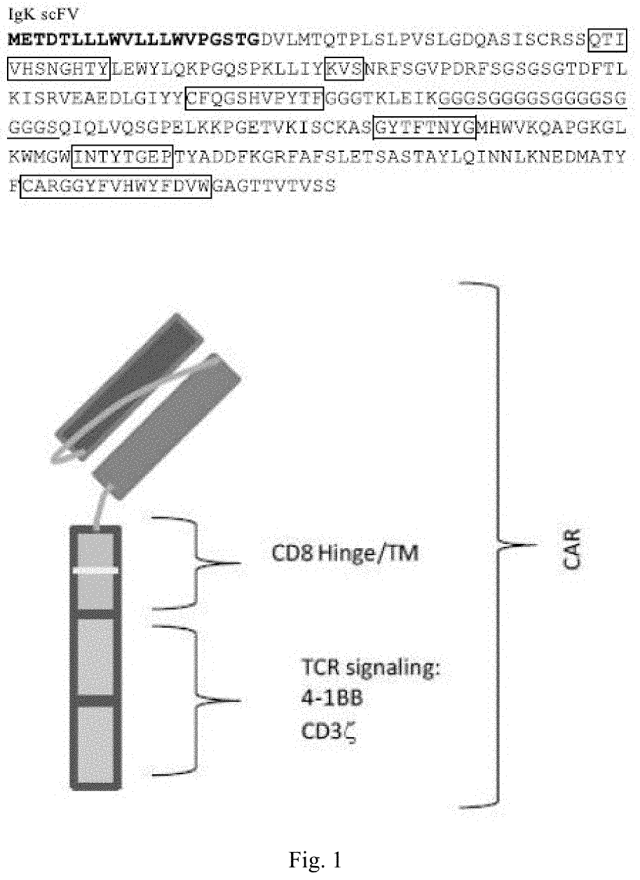

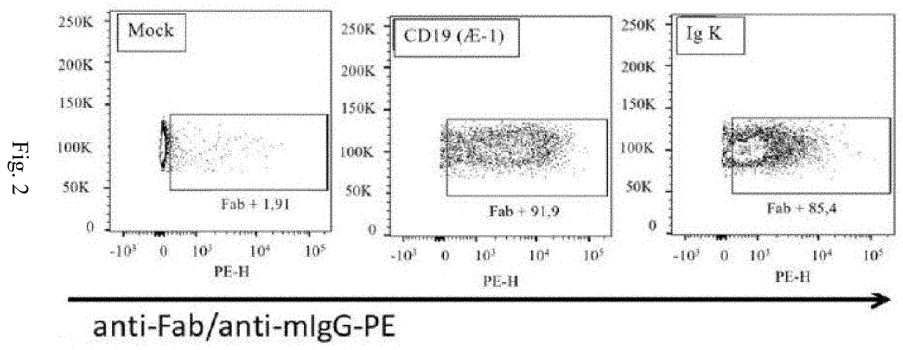

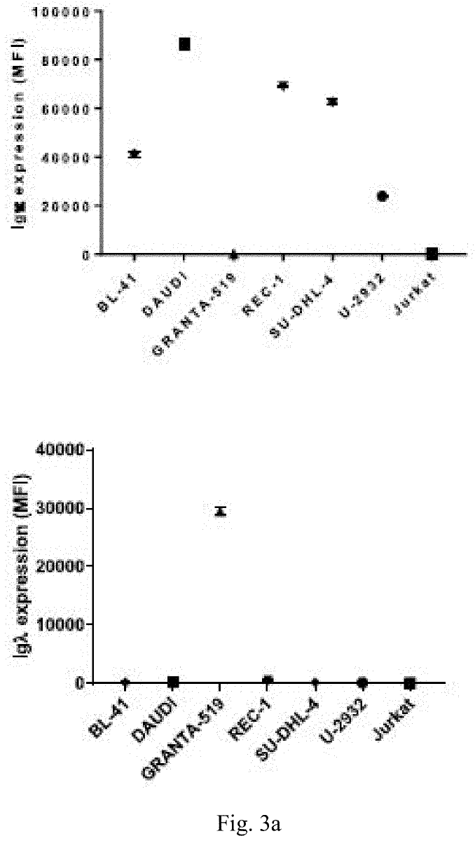

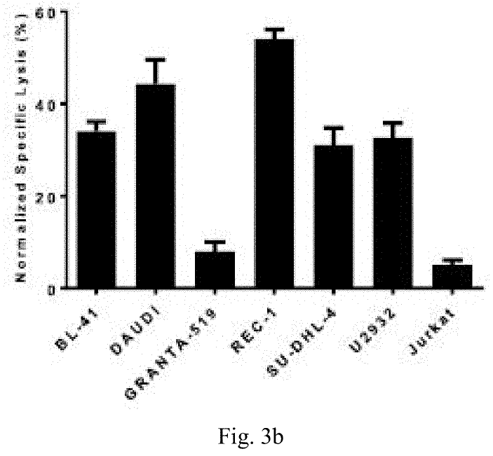

As used herein, IGK CAR means a CAR specific for IgKappa wherein the intracellular domain comprises a CD3ζ-signaling domain and a 4-1BB costimulatory domain as visualized in FIG. 1 and FIG. 4c.

As used herein, CD19 CAR means a CAR specific for CD19 wherein the intracellular domain comprises a CD3ζ-signaling domain and a 4-1BB costimulatory domain (visualized in FIG. 4c).

As used herein, Kz means a CAR specific for IgKappa wherein the intracellular domain comprises or consists of a CD3ζ-signaling domain, i.e. the intracellular domain does not comprise a functional costimulatory domain (visualized in FIG. 4c).

As used herein, 19z means a CAR specific for CD19 wherein the intracellular domain comprises or consists of a CD3ζ-signaling domain, i.e. the intracellular domain does not comprise a functional costimulatory domain (visualized in FIG. 4c).

As used herein, KBB means a CAR specific for IgKappa wherein the intracellular domain comprises or consists of a 4-1BB costimulatory domain, i.e. the intracellular domain does not comprise a CD3ζ-signaling domain (visualized in FIG. 4c).

As used herein, 19BB means a CAR specific for CD19 wherein the intracellular domain comprises or consists of a 4-1BB costimulatory domain, i.e. the intracellular domain does not comprise a CD3ζ-signaling domain (visualized in FIG. 4c).

As used herein, 19z-KBB means the combinatorial CAR comprising 19z and KBB (visualized in FIG. 4a).

As used herein, Kz-19BB means the combinatorial CAR comprising Kz and 19BB (visualized in FIG. 4a).

### DETAILED DESCRIPTION

Chimeric antigen receptor (CAR) based immunotherapy is recently FDA approved for treatment of B-cell acute leukemia and diffuse large B-cell lymphoma. This is mainly due to the success of CAR T cells targeting B-lymphocyte antigen CD19, which has led to astonishing results in clinical trials. Considering that all B cells express CD19 antigen, CAR T cells eliminate all B cells, including non-malignant B cells. Therefore, the patients suffer from impaired humoral immune response, specifically B-cell aplasia and hypogammaglobulinemia, which might increase susceptibility to severe infections. Another problem is related to the target itself. Accumulation of data demonstrates the possibility of immune escape by down regulation of CD19 or alternative splicing variant leading to resistance to CD19 CAR T cells. There is therefore a need for alternative targets.

B-cells express a form of transmembrane immunoglobulins (Igs) in their cell membrane. These immunoglobulins may bind extracellular antigens and deliver a signal into the B-cell. Accordingly, such immunoglobulins are known as B-cell receptors (BCRs). Like other antibodies, the BCRs comprise heavy chains and light chains, each chain comprising a variable domain and a constant domain.

In mammals, there are two types of light chains; the kappa light chain and the lambda light chain. Each B-cell and each BCR will comprise either kappa light chains or lambda light chains. Accordingly, clonal populations of B-cells will also express BCRs comprising either the kappa light chain or the lambda light chain. This allows for targeting of clonal populations of B-cells, e.g. malignant B-cell populations, based on recognition of the BCR comprising kappa light chain.

This present disclosure relates to combinatorial chimeric antigen receptors (cCARs) with antigen binding domains specific for CD19 and IgKappa. Thus, the cCAR may direct cytotoxic immune cells to malignant B-cells expressing BCRs comprising the kappa light chain. Accordingly, cytotoxic immune cells expressing the cCAR may be used in treatment of Ig kappa expressing B-cell cancers, e.g. B-cell acute lymphoblastic leukemia (ALL), chronic lymphocytic leukemia (CLL) and B-cell lymphoma. The present disclosure provides nucleic acid molecules encoding such CARs and vectors containing them which may be used to modify immune effector cells to express the combinatorial CAR. In one particular embodiment, the combinatorial CARs comprise a novel antigen-binding protein specific for IgKappa.

In particular, the antigen-binding domains of the combinatorial CAR are capable of binding specifically to CD19 and IgKappa (more particularly when the cCAR is expressed on the surface of an immune effector cell). Specific binding may be distinguished from non-specific binding to a non-target antigen (in this case an antigen other than CD19) or non-specific binding to a non-target IgLambda.

Thus, an immune effector cell expressing the combinatorial CAR according to the present disclosure is redirected to bind specifically to and exhibit cytotoxicity to (e.g. kill) a IgKappa and CD19-expressing target cell. Alternatively expressed, the immune effector cell is modified to redirect cytotoxicity towards target cells expressing CD19 and IgKappa.

Taking into account that B-cell lymphomas and CLL cells have a clonally restricted expression of Ig light chains, IgKappa positive tumor cells can be targeted while sparing normal IgLambda positive B-cells. Hence, CAR T cells with an antigen binding domain specific for IgKappa could provide lower on-target toxicity than anti-CD19 CAR T cells and would be expected to improve the life quality of the patients. As demonstrated herein, the efficacy and specificity of IGK CAR T cells showed that the concept can be an efficient alternative to CD19 CAR T cells.

Additionally, the disclosure addresses the inhibition of IGK CAR T cells' killing efficacy by free IgGs in the serum. It has been found that IgKappa CAR cytotoxic activity is negatively affected in the presence of human serum (HS). This is visualized in FIG. 4b.

The results herein demonstrate that a combinatorial CAR can be utilized to overcome the in vitro inhibition caused by the presence of free IgGs. This is achieved by designing combinatorial CARs comprising two different scFv (antigen binding domains), wherein one of them is specific for CD19.

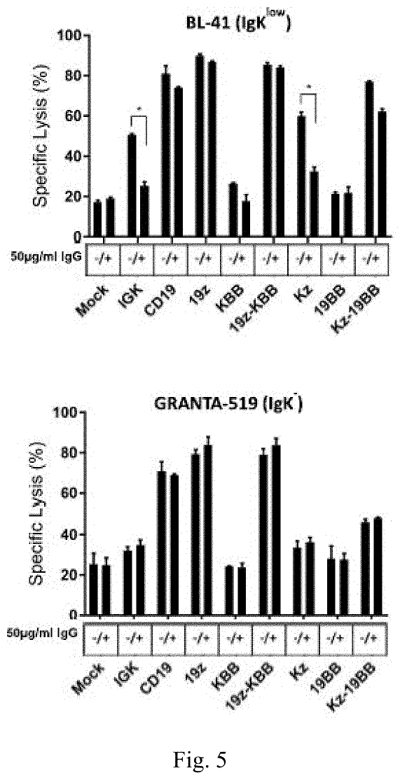

Cytotoxic activity of combinations was assessed against BL-41 and GRANTA-519 by BLI-based killing assay after 10 hours of co-culture. The results demonstrated that against BL-41, IGK CAR T and Kz were significantly affected by the presence of IgGs but CARs with 19z-KBB and Kz-19BB combinations were not significantly affected. This indicates that combinatorial CARs can be potent alternatives to second generation CARs with better precision. Overall, designs with only costimulatory domains were only able to demonstrate low potency in the cytotoxic assay. This demonstrate that the primary domain responsible for the killing activity is the CD3ζ signaling domain. FIG. 5 shows that CAR T cells with different combinations of intracellular domains (e.g. CD3ζ signaling domain and the intracellular costimulatory domain) have different cytotoxic potential against lymphoma cell lines (BL-41 and GRANTA-519).

IGK CAR is only potent against IgKappa positive target cells (BL-41) and no cytotoxic activity was observed against IgKappa negative cells, i.e. the IgLambda positive cells (Granta-519). Two different combinatorial CARs were tested, Kz-19BB and 19z-KBB.

As demonstrated in FIG. 5, the combinatorial CAR, Kz-19BB, demonstrates a similar selectivity as IGK CAR. Still as potent against IgKappa positive target cells and less harmful to IgKappa negative cells than CD19 CAR. However, surprisingly it was demonstrated that the 19z-KBB is similarly devastating to the Granta-519 cells, thus the risk that the entire normal B-cell population in the patient may be eradicated during the treatment is similar to the CD19 CARs of the prior art.

Additionally, the same CAR constructs were tested in the presence of serum purified immunoglobulins (IgG), to see if IgG-serum inhibited the lysis. Both IGK, KBB and Kz CAR T cells were inhibited in the presence of very low concentration of IgG and killing efficiency were significantly reduced, whereas CD19 CARs was not affected by the IgG at all, proving the effect is specific to IGK CAR T cells. Furthermore, combinatorial CARs were not affected by the same IgG concentrations to the extent of IGK CAR, indicating that a combinatorial CAR may be useful for balancing potency and specificity.

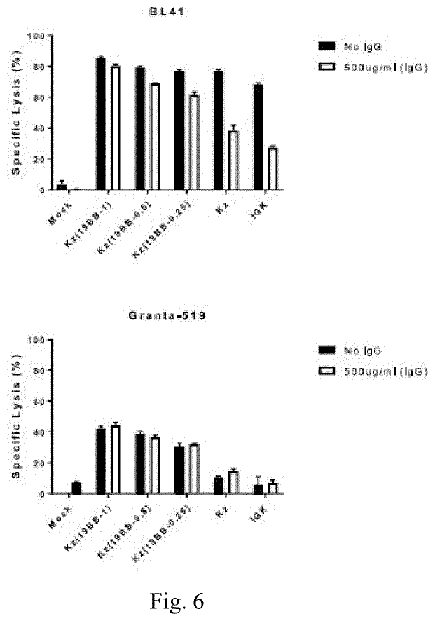

It is further demonstrated that the cytotoxic potential of the combinatorial CAR is adjustable by increasing the relative amount of nucleic acids encoding the 19BB used for transducing the cells (see FIG. 6). In the presence of high concentration of IgG classic IGK CAR activity is inhibited. However, the effect is recovered with increasing concentration of 19-BB part of the combinatorial CAR. The higher the expression levels of CD19 component, the more the final design becomes CD19 CAR-like. Similar to this observation, the higher the concentration of CD19 component the more IgKappa negative CD19 positive killing we observed.

Accordingly, the disclosure demonstrate that it is possible to adapt the combinatorial CAR T-cells efficiency to the need of the patients. As substantiated by the in vitro results, one can balance between cytotoxic potential and specificity to eliminate the malignant B-cell portion and save some of the healthy B cells to reduce the harmful impact of classic CD19 CAR T cells on general humoral immune response. This can be achieved by transducing cytotoxic immune cells by nucleic acids encoding the CARs wherein the relative fraction of nucleic acids encoding Kz is increased compared to 19BB (see FIG. 6). This can also be achieved in other ways e.g. adjusting the expression levels based on the nucleic acid constructs.

Provided herein, is also a novel antigen binding protein specific for IgKappa under physiological conditions. When targeting disease-causing cells in vivo, it is of great importance to have alternative targeting molecules available. If one treatment loses its efficacy or triggers unwanted immune responses, another treatment may not cause the same problem. Two antigen binding proteins specific for a target molecule do not necessarily bind to the same epitope. This is particularly important for tumor targeting, because cancer cells may mutate their epitopes and evade recognition. Accordingly, alternative antigen binding proteins suitable for tumor targeting are needed.

Antibodies, such as IgGs, comprise two identical antigen binding domains. These domains tend to form a three-dimensional structure under physiological conditions which are able to bind a target molecule. Some antigen binding domains are robust enough to essentially keep their three-dimensional structure if connected to other molecules. In some cases, the antigen binding domain can thus keep its target specificity and/or target affinity even if fused to unrelated protein domains.

Close association of the two amino acid sequences forming the antigen binding domain specific for human IgKappa is needed. This can be achieved in many ways, but the most convenient one may be to connect them by a flexible peptide linker from the C-terminal of one sequence to the N-terminal of the other sequence. Such linkers are well known for skilled persons, and they usually comprise a high fraction of glycine and/or serine residues. Antigen binding proteins comprising such linkers can be formed by recombinant expression (e.g. as single chain Fv-fragments, scFv). Alternatively, the two amino acid sequences may be connected by disulfide bridges in the same way as ordinary light and heavy chains in antibodies. Connection via leucine zippers may also be possible.

Each of the two amino acid sequences in the antigen binding protein herein comprise three complementarity-determining regions (CDRs) flanked by framework regions according to well-known general antibody structure.

The CDRs may all contribute to the specificity for IgKappa.

The three CDRs in SEQ ID 1 as well as in SEQ ID 3 are represented by

(SEQ ID 5)
QTIVHSNGHTY
(SEQ ID 6)
KVS
(SEQ ID 7)
CFQGSHVPYTF

The three CDRs in SEQ ID 2 as well as in SEQ ID 4 are represented by

(SEQ ID 8)
GYTFTNYG
(SEQ ID 9)
INTYTGEP
(SEQ ID 10)
CARGGYFVHWYFDVW

The length and sequence of the framework regions are believed to be important for configuration of the CDRs to form an antigen binding protein specific for IgKappa. However, some conservative amino acid substitution is believed to be tolerated in SEQ ID 1/SEQ ID 3 and SEQ ID 2/SEQ ID 4 without losing the specific target affinity.

In particular, the antigen binding protein specific for IgKappa may comprise SEQ ID 1 or sequences more than 95% (96%, 97%, 98% or 99%) identical to the amino acid sequence SEQ ID 1 provided any difference to SEQ ID 1 is in the form of conservative amino acid substitution.

In particular, the antigen binding protein specific for IgKappa may comprise SEQ ID 2 or sequences more than 95% (96%, 97%, 98% or 99%) identical to the amino acid sequence SEQ ID 2 provided any difference to SEQ ID 2 is in the form of conservative amino acid substitution.

In particular, the antigen binding protein specific for IgKappa may comprise SEQ ID 3 or sequences more than 95% (96%, 97%, 98% or 99%) identical to the amino acid sequence SEQ ID 3 provided any difference to SEQ ID 3 is in the form of conservative amino acid substitution.

In particular, the antigen binding protein specific for IgKappa may comprise SEQ ID 4 or sequences more than 95% (96%, 97%, 98% or 99%) identical to the amino acid sequence SEQ ID 4 provided any difference to SEQ ID 4 is in the form of conservative amino acid substitution.

As used herein, conservative amino acid substitution includes the very highly conserved substitutions, highly conserved substitutions and conserved substitutions according to Table 1.

TABLE 1

| | | Highly Conserved | Conserved |
|---|---|---|---|
| | Very Highly - | Substitutions | Substitutions |
| Original | Conserved | (from the | (from the |
| Residue | Substitutions | Blosum90 Matrix) | Blosum65 Matrix) |
| Ala | Ser | Gly, Ser, Thr | Cys, Gly, Ser, Thr, Val |
| Arg | Lys | Gln, His, Lys | Asn, Gln, Glu, His, Lys |
| Asn | Gln; His | Asp, Gln, His, Ly, Ser, Thr | Arg, Asp, Gln, Glu, His, |
| | | | Lys, Ser, Thr |
| Asp | Glu | Asn, Glu | Asn, Gln, Glu, Ser |
| Cys | Ser | None | Ala |
| Gln | Asn | Arg, Asn, Glu, His, Lys, Met | Arg, Asn, Asp, Glu, His, |
| | | | Lys, Met, Ser |
| Glu | Asp | Asp, Gln, Lys | Arg, Asn, Asp, Gln, His, Lys, Ser |
| Gly | Pro | Ala | Ala, Ser |
| His | Asn; Gln | Arg, Asn, Gln, Tyr | Arg, Asn, Gln, Glu, Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, Ser |
| Met | Leu; Ile | Gln, Ile, Leu, Val | Gln, Ile, Leu, Phe, Val |
| Phe | Met; Leu; Tyr | Leu, Trp, Tyr | Ile, Leu, Met, Trp, Tyr |
| Ser | Thr | Ala, Asn, Thr | Ala, Asn, Asp, Gln, Glu, |
| | | | Gly, Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, Thr |

The antigen binding protein may be recombinantly produced in by methods well known for skilled persons. E.g. by conventional expression vectors in mammalian cell lines like Hek-293 or CHO.

The present disclosure provides an antigen binding protein specific for IgKappa comprising a first amino acid chain and a second amino acid chain;

wherein the first chain comprises the amino acid sequence SEQ ID 1, or sequences more than 95% identical to the amino acid sequence SEQ ID 1 provided any difference to SEQ ID 1 is in the form of conservative amino acid substitution;
and
wherein the second chain comprises the amino acid sequence SEQ ID 2, or sequences more than 95% identical to the amino acid sequence SEQ ID 2 provided any difference to SEQ ID 2 is in the form of conservative amino acid substitution.

The present disclosure provides an antigen binding protein specific for IgKappa comprising a first amino acid chain and a second amino acid chain;

wherein the first chain comprises the amino acid sequence SEQ ID 3, or sequences more than 95% identical to the amino acid sequence SEQ ID 3 provided any difference to SEQ ID 3 is in the form of conservative amino acid substitution;
and
wherein the second chain comprises the amino acid sequence SEQ ID 4, or sequences more than 95% identical to the amino acid sequence SEQ ID 4 provided any difference to SEQ ID 4 is in the form of conservative amino acid substitution.

The present disclosure provides an antigen binding protein specific for IgKappa comprising a first amino acid chain and a second amino acid chain; wherein the first chain comprises three CDR sequences represented by SEQ ID 5, 6 and 7 and wherein the second chain comprises three CDR sequences represented by SEQ ID 8, 9 and 10.

The present disclosure provides an antigen binding protein specific for IgKappa comprising a first amino acid chain and a second amino acid chain; wherein the first chain comprises the amino acid sequence SEQ ID 1 and wherein the second chain comprises the amino acid sequence SEQ ID 2.

The present disclosure provides an antigen binding protein specific for IgKappa comprising a first amino acid chain and a second amino acid chain; wherein the first chain comprises the amino acid sequence SEQ ID 3 and wherein the second chain comprises the amino acid sequence SEQ ID 4.

The present disclosure also provides nucleic acids (e.g. RNA and DNA) encoding the antigen binding proteins mentioned above. The present disclosure also provides CARs comprising the antigen binding proteins mentioned above.

It is not trivial to obtain robust antigen binding proteins with specific target affinity. However, it is found that the antigen binding protein disclosed herein may retain its target specificity and/or target affinity even when expressed in a chimeric antigen receptor (CAR) construct by T-cells. Furthermore, such CARs may be able to deliver a signal into immune effector cells upon binding of IgKappa positive target cells. Accordingly, immune effector cells, like T-cells and NK-cells, expressing these CARs in their cell membrane may thus provide cytotoxicity to B-cells expressing IgKappa.

In a particular embodiment, immune effector cells may be genetically modified to express the CARs disclosed herein. This can be achieved in many ways e.g. transduction of a viral vector comprising a nucleic acid encoding a CAR or transduction of mRNA encoding a CAR. The lymphocytes can be activated and/or expanded before or after the genetic modification using methods well known to a skilled person.

The CARs herein comprise an extracellular domain, a transmembrane domain and an intracellular domain, and they may deliver a signal into immune effector cells if expressed in their cell membrane.

As used herein, “extracellular domain”, means the part of the CAR facing the extracellular environment when expressed in the cell membrane of an immune effector cell. The extracellular domain comprises an antigen binding protein and optionally a hinge domain. Suitable hinge domains are well known for skilled persons. In particular, hinge domains from CD8α, CD28, IgGCH2,3 may be used.

As used herein, “transmembrane domain”, means the part of the CAR which tend to be embedded in the cell membrane when expressed by an immune effector cell. Suitable transmembrane domains are well known for skilled persons. In particular, transmembrane domains from CD8α or CD28 or ICOS may be used.

As used herein “intracellular domain” refers to the part of the CAR located inside the immune effector cell that participates in conveying the signal upon binding of the target. The signal may contribute to activation, cytokine production, proliferation and/or cytotoxic activity or inhibition (iCAR). A variety of signaling domains are known, and they can be combined and tailored to fit the endogenous signaling machinery in the immune effector cells.

As used herein, an intracellular signaling domain is a “signal 1” domain like the signaling domains obtainable from CD3ζ, FcR-γ, CD3ε etc. In general, it is believed that “signal 1”-domains (e.g. CD3ζ signaling domain represented by SEQ ID 12) convey a signal upon antigen binding. As used herein, intracellular costimulatory domains means the “signal 2”-domains (e.g. 4-1BB signaling domain represented by SEQ ID 13) believed to subsequently convey a signal via costimulatory molecules. The “signal 2” is essential for the maintenance of the signal and the survival of the cells, if absent (1^{st} generation CAR), the redirected cell will be as efficient in killing and in early cytokines release, but will become exhausted afterwards. Examples of such commonly used “signal 2” domains include 4-1BB signaling domain, CD28 signaling domain, OX40 signaling domain and ICOS signaling domain.

The CARs may be recombinantly produced by methods well known for skilled persons, but for therapeutic use, T-cells or natural killer cells are preferred host cells. In particular, as exemplified herein, primary T-cells may be transduced by electroporation with mRNA encoding the CARs.

For efficient expression, a conventional leader peptide (i.e. signal peptide or L-chain) may be introduced N-terminally for facilitating location in the plasma membrane. The leader peptide is believed to be trimmed off and will likely not be present in the functional CAR.

It is found that soluble IgGs may reduce the cytotoxicity of the immune effector cells herein. This may have a negative impact if the immune effector cells is administered intravenously, as IgGs are found in substantial amounts in blood, serum and extracellular fluids. Without being bound by theory, it may be that IgGs exhaust the immune effector cells expressing IgKappa CARs. Surprisingly, by expressing a CAR specific for IgKappa together with a CAR specific for CD19, this problem may be avoided. This is visualized in FIG. 4b Immune effector cells expressing both these types of CARs may be an improved alternative (based on cytotoxicity and/or specificity) to conventional therapy based on a single CAR specific for CD19 only.

Furthermore, is found that immune effector cells expressing a CAR specific for IgKappa together with a CAR specific for CD19 may have significantly reduced specificity for IgKappa positive B-cells. Surprisingly, when the CAR specific for CD19 comprised CD3ζ-signaling domain, and the CAR specific for IgKappa comprised the 4-1BB signaling domain, the specificity was improved. This is visualized in FIG. 4b, FIG. 5 and FIG. 6.

SEQUENCES:
SEQ ID 1 (slightly shorter VL chain with CDrs boxed)

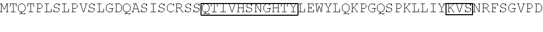

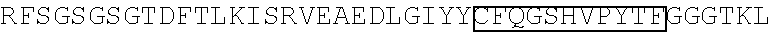

SEQ ID 2 (slightly shorter VH chain with CDRs boxed)

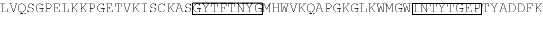

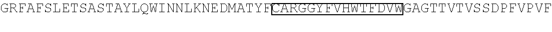

LPAKPTTTPAPRPP
SEQ ID 3 (VL chain with CDRs boxed)

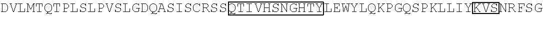

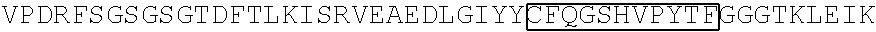

SEQ ID 4 (VH chain with CDRs boxed)

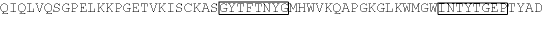

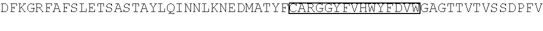

PVFLPAKPTTTPAPRPPTPA
SEQ ID 5 (CDR1 VL)
QTIVHSNGHTY
SEQ ID 6 (CDR2 VL)
KVS
SEQ ID 7 (CDR3 VL)
CFQGSHVPYTF
SEQ ID 8 (CDR1 VH)
GYTFTNYG
SEQ ID 9 (CDR2 VH)
INTYTGEP
SEQ ID 10 (CDR3 VH)
CARGGYFVHWYFDVW
SEQ ID 11 (CD8α transmembrane domain)
IYIWAPLAGTCGVLLLSLVIT
SEQ ID 12 (CD3ζ signaling domain)
RVKFSRSADAPAYQQGQNQLYNELNLGRREEYDVLDKRRGRDPEMGGKPQRRKNPQEGLYNE
LQKDKMAEAYSEIGMKGERRRGKGHDGLYQGLSTATKDTYDALHMQALPPR
SEQ ID 13 (4-1BG signaling domain)
KRGRKKLLYIFKQPFMRPVQTTQEEDGCSCRFPEEEEGGCEL

### EXAMPLES

### Example 1

### Car Expression:

Retroviral particles of pSFG.aCD37HCH2CH3-CD28OXZ (encoding the 3rd generation CAR) were prepared as follows: HEK-Phoenix (HEK-P, our collection) were grown in DMEM (PAA) supplemented with 10% HyClone FCS (HyClone) and 1% antibiotic-antimicotic (penicillin/streptomycin, P/S, PAA). Viral particles were produced using HEK-P cells transfected using Fugene-6 (Roche) with retroviral packaging vectors and the expression vector. After 24 hours of incubation at 37° C., medium was replaced with DMEM 1% FCS and cells were incubated at 32° C. Supernatants were harvested after 24 and 48 hours.

PBMCs isolated from healthy donors were cultured and activated in X-VIVO™ 20 media supplemented with 5% human serum and 100 U/ml IL2 (R&D Systems) for 48 hours in a 24 well plate pre-coated with anti-CD3ζ (OKT-3) and anti-CD28 antibodies (BD Biosciences). After two days of culture PBMCs were harvested. Spinoculation of T cells from PBMC was performed with 1 ml of retroviral supernatant in a 12-well culture non-treated plate (Nunc A/S) pre-coated with retronectin (20 mg/mL, Takara Bio.). Spinoculation was repeated once, 1 day after the 1st spinoculation. On day 7 post-transduction, PBMCs were used for experiments. The same protocol was followed to express the 2nd generation CAR, except that it was cloned into an MP71-gateway adapted vector (see Walchli et al, 2011).

mRNA was prepared following the standard protocol:

### Reagents

TABLE 3

| Name | Product Reference |
|---|---|
| CutSmart Buffer | NEB, B7204S |
| MfeI-HF | NEB, R3589L |
| Nuclease Free Water (NFW) | Sigma, W4502 |
| NotI-HF | NEB, R3189L |
| Flash Gel DNA Cassette | Lonza, 57023 |
| Flash Gel Loading Dye | Lonza, 50463 |
| Flash Gel DNA Marker 100-4000 bp | Lonza, 50473 |
| Wizard SV Gel and PCR Clean-Up System | Promega, A9281 |
| Molecular AccuGENE | Lonza, 51200 |
| RiboMAX Large Scale RNA Production System | Promega, P1300 |
| ARCA | Tri-Link, N-703 |
| MEGAclear | Ambion, AM1908 |
| Flash Gel RNA Cassette | Lonza, 57027 |
| Flash Gel RNA Marker | Lonza, 50577 |
| Formaldehyde Sample Buffer | Lonza, 50569 |
| Ethidium Bromide | Invitrogen, 15585-011 |
| Gel Loading Dye | NEB, B70245 |
| 1 kb DNA ladder | NEB, N3232L |

TABLE 4
LINEARIZATION OF TEMPLATE DNA

| Reagent | Volume | |
|---|---|---|
| MQ water (to 500 μl) | X | μl |
| Cut smart buffer (x10) | 50 | μl |
| MfeI-HF | 15 | μl |
| Plasmid DNA (100 μg) | Y | μl |
| TOTAL | 500 | μl |

100 μg Plasmid DNA was digested, enough for 500 μl mRNA synthesis (40 μg/200 μl synthesis) by incubation for 4 hrs at 37° C., followed by:
1. Inactivation of enzyme activity by placing tube in heating block, 65° C., 15 mins.
2. Proceeding with purification (or storage at −20° C.).

Purification of linearised template DNA:
1. Wizard SV Gel and PCR Clean-Up System by Promega were used.
2. An equal volume of Membrane Binding Solution was added to the DNA and mixed well.

### Binding of DNA

3. SV Minicolumn was inserted into Collection tube.
4. Dissolved mixture (1000 μl) was transferred to the Minicolumn assembly ×2, then incubated at RT for 1 minute.
5. Minicolumn assembly was centrifuged at 16,000×g for 1 min Flow-through was discarded and the Minicolumn re-inserted into the Collection Tube.

### Washing

6. 700 μl Membrane Wash Solution (with EtOH added) was added to the Minicolumn. The Minicolumn assembly was centrifuged at 16,000×g for 1 min. Flow-through was discarded and the Minicolumn reinserted into the Collection Tube.
7. Step 6 was repeated with 500 μl Membrane Wash Solution. Centrifugation was performed at 16,000×g for 1 min Flow-through was discarded, and the column centrifuged for another 5 min at 16,000×g.
8. The Minicolumn was carefully transferred to a clean 1.5 ml microcentrifuge tube.
9. 50 μl NFW was added to the Minicolumn Minicolumn was then incubated at RT for 1 min, then centrifuged at 16,000×g for 1 min
10. Minicolumn was discarded.
11. DNA concentration was measured using NanoDrop ND-1000 Spectrophotometer.

### In Vitro Transcription

TABLE 5
MRNA SYNTHESIS

| Reagent | Volume | |
|---|---|---|
| Nuclease Free Water (to 100 μl) | X | μl |
| rATP | 7.5 | μl |
| rCTP | 7.5 | μl |
| rUTP | 7.5 | μl |
| rGTP | 2.3 | μl |
| ARCA Cap | 9.0 | μl |
| T7 Buffer* | 20.0 | μl |
| Template DNA (50 ng/μl) | Y | μl |
| T7 Enzyme Mix | 10.0 | μl |
| Total | 100 | (μl) |

*Buffer was heated to 37° C. to dissolve precipitated material and mixed regularly for complete dissolution. Buffer was kept at RT while setting up the reaction.

1. Mixture was mixed with a pipette and incubated at 37° C. for 5 hrs.
2. 5 μl RQ1 RNase-free DNase (1 U/μl) (Promega) was added per 100 μl reaction volume, mixed well and incubated for another 20 mins at 37° C.
3. Mixture was stored at −20° C. overnight.
   mRNA Isolation

mRNA was isolated using MEGAclear KIT from Ambion.

If sample volume was less than 100 μl, sample was brought to 100 μl with Elution Solution and mixed gently.
1. 350 μl of Binding Solution was added per 100 μl sample and mixed gently.
2. 250 μl 100% ethanol was added per 100 μl sample and mixed gently.
3. Sample was applied to the filter:
   a) A Filter Cartridge was inserted into a Collection and Elution Tube.
   b) The RNA mixture was applied to the Filter Cartridge.
   c) The Filter Cartridge was centrifuged at 10,000-15,000×g for 1 min
   d) The flow-through was discarded.
4. The Filter Cartridge was washed with 3×500 μl Wash Solution.
   a) 500 μl Wash Solution was applied to the Filter Cartridge. This was then centrifuged at 15,000×g for 1 min and the flow-through discarded.
   b) Step ‘a’ was repeated twice.
   c) A final centrifugation step was performed to remove the last traces of Wash Solution (1 min for 15,000×g).
5. RNA was eluted from the filter with 50 μl Elution Solution by centrifugation T-cell electroporation was carried out as in Wälchli et al, 2011.

Briefly, IGK CAR mRNA was transferred into PBMC derived T-cells isolated from a healthy donor by electroporation. Cells were grown for 12-24 hours after electroporation and expression levels of IGK CAR were detected by flow cytometry and compared to the expression of a validated construct (CD19 CAR, clone fmc63). To this end, a biotinylated anti-mouse Fab antibody and a secondary antibody Streptavidin conjugated to PE were used following this protocol: Anti-Fab staining: 200 μL isolated T-cells were washed once, resuspended in 10 μL anti-Fab antibody (Goat F(ab′)₂ Anti-Mouse IgG F(ab′)2 (Biotin), Abcam 98657) and incubated for 20 min at RT. They were then washed once more. Added 5 μL Streptavidin-PE in Flow buffer, incubated for 10 min at RT. Cells were washed a final time, then resuspended in 180 μl Flow Buffer (PBS+2% FCS) and expression analysed by flow cytometry.

### Example 2

Different cell lines were evaluated for their IgKappa and IgLambda expression profile. Anti-IgK and Anti-IgL staining: 200 μL (ca 0.2 M cells) of the indicated B-cell line were washed once, resuspended in 10 μL anti-human-IgK antibody APC-labeled (Biolegend, 316509) and 10 μL anti-human-IgL antibody FITC-labeled (Biolegend, 316606) and incubated for 20 min at RT. They were then washed once more and resuspended in 200 μl Flow Buffer (PBS+2% FCS) and expression analyzed by flow cytometry.

IgK-CAR activity was tested in a killing assay. Redirected T-cells from healthy donors (with CD19 fmc63, IgK or mock) were incubated with different B cell lines positive for IgKappa positive (DAUDI, SU-DHL-4, U2932, REC-1 and BL-41) and control cell lines; IgLambda positive (Granta-519) and both IgKappa and IgLamda negative (Jurkat). The target cells have been previously permanently transformed to express luciferase. Upon incubation with the substrate luciferin, activity can be detected under a luminometer. More precisely, Luciferase-expressing tumor cells were counted and resuspended at a concentration of 3×105 cells/mL. Cells were given Xenolight D-Luciferin potassium salt (75 μg/ml; Perkin Elmer) and were placed in 96-well white round bottoms as 100 μl cells/well in triplicates. Subsequently, effector cells were added as 1:10 effector-to-target (E:T) ratio. In order to determine baseline and maximal killing capacity, three wells were left with only target cells and another three with target cells in 1% Triton™ X-100 (Sigma-Aldrich). Cells were incubated at 37° C. for 2 hours. Bioluminescence (BLI) was measured with a luminometer (VICTOR Multilabel Plate Reader) as relative light units (RLU). Target cells that were incubated without any effector cells were used to determine baseline spontaneous death RLU in each time point. Triplicate wells were averaged and lysis percentage was calculated using following equation: % specific lysis=100×(spontaneous cell death RLU−sample RLU)/(spontaneous death RLU−maximal killing RLU). Plotting and statistical analysis were performed using GraphPad prism software (La Jolla, Calif. USA).

These experiments show that IGK CAR T cells were as potent to recognize and kill IgKappa+ B-cell lymphoma as CD19CAR T cells.

### Example 3

In terms of IgG inhibition, 19z-KBB has proved to be unaffected by any concentration of IgG. As CD3ζ is a very powerful signaling domain, the 19z CAR may kill IgKappa+ as well as IgLambda positive cells. However, the cCAR 19z-KBB might still be an alternative to regular CD19 CAR treatment. On the other hand, Kz-19BB is less effected by the presence of IgG and seems to be performing better with respect to specificity compared to regular second generation IGK CAR.

1. A cytotoxic immune cell expressing at least two CARs in the cell membrane, the CARs comprising
i. a first CAR specific for CD19 comprising an extracellular domain, a transmembrane domain and an intracellular costimulatory domain; and
ii. a second CAR specific for IgKappa comprising an extracellular domain, a transmembrane domain and an intracellular signaling domain.

2. The cell according to claim 1, wherein the intracellular domain of the first CAR specific for CD19 does not comprise a functional intracellular signaling domain (“signal 1” domain).

3. The cell according to claim 1, wherein the intracellular domain of the second CAR specific for IgKappa does not comprise a functional costimulatory domain (“signal 2” domain).

4. The cell according to claim 1, wherein the intracellular domain of the first CAR specific for CD19 comprises a 4-1BB signaling domain, and wherein the intracellular domain of the second CAR specific for IgKappa comprises a CD3ζ-signaling domain.

5. The cell according to claim 1, wherein the cell is a CD8+ T-cell or an NK cell.

6. The cell according to claim 1, wherein the extracellular domain of the second CAR specific for IgKappa comprises a first amino acid chain and a second amino acid chain;
wherein the first chain comprises the amino acid sequence SEQ ID 1, or a sequence more than 95% identical to the amino acid sequence SEQ ID 1 provided any difference to SEQ ID 1 is in the form of conservative amino acid substitution;
and
wherein the second chain comprises the amino acid sequence SEQ ID 2, or a sequence more than 95% identical to the amino acid sequence SEQ ID 2 provided any difference to SEQ ID 2 is in the form of conservative amino acid substitution.

7. The cell according to claim 1, wherein the extracellular domain of the second CAR specific for IgKappa comprises a first amino acid chain and a second amino acid chain;
wherein the first chain comprises the amino acid sequence SEQ ID 3, or a sequence more than 95% identical to the amino acid sequence SEQ ID 3 provided any difference to SEQ ID 3 is in the form of conservative amino acid substitution;
and
wherein the second chain comprises the amino acid sequence SEQ ID 4, or a sequence more than 95% identical to the amino acid sequence SEQ ID 4 provided any difference to SEQ ID 4 is in the form of conservative amino acid substitution.

8. The cell according to claim 1, wherein the extracellular domain of the second CAR specific for IgKappa comprises a first amino acid chain represented by SEQ ID 3 and a second amino acid chain represented by SEQ ID 4.

9. The cell according to claim 6, wherein the first chain comprises three CDR sequences represented by SEQ ID 5, 6 and 7 and wherein the second chain comprises three CDR sequences represented by SEQ ID 8, 9 and 10.

10. A pharmaceutical composition suitable for intravenous, intraperitoneal or subcutaneous administration comprising a therapeutic amount of the cells according to claim 1.

11. (canceled)

12. A method of treating B-cell cancers comprising administering to a subject in need thereof the cytotoxic immune cell according to claim 1.

13. A composition comprising nucleic acids encoding the CARs as defined in claim 1.
